(19) United States
(12) Patent Application Publication
(10) Pub. No.: US 2023/0260639 A1
(43) Pub. Date: Aug. 17, 2023

(54) INFORMATION PROCESSING DEVICE, INFORMATION PROCESSING METHOD, AND INFORMATION PROCESSING PROGRAM

(71) Applicant: FUJIFILM CORPORATION, Tokyo (JP)

(72) Inventors: Haruyasu NAKATSUGAWA, Kanagawa (JP); Yasuhisa KANEKO, Kanagawa (JP); Kenji NAGAMIYA, Kanagawa (JP); Tomohide HIRAGAMI, Kanagawa (JP); Yasuyuki HOSONO, Kanagawa (JP); Nobuya KITAMURA, Kanagawa (JP)

(21) Appl. No.: 18/304,371

(22) Filed: Apr. 21, 2023

Related U.S. Application Data
(63) Continuation of application No. PCT/JP2021/039888, filed on Oct. 28, 2021.

(30) Foreign Application Priority Data
Oct. 29, 2020 (JP) 2020-181879

(51) Int. Cl. G16H 40/20; B08B 7/00

(52) U.S. Cl. CPC G16H 40/20; B08B 7/00

(57) ABSTRACT

An information processing device comprising at least one processor, wherein the at least one processor is configured to: acquire presence information that is information indicating a state of presence of a person in each of a plurality of regions, for each region; and generate, for each of the plurality of regions, cleaning information for instructing cleaning of the region based on the presence information.

### CROSS REFERENCE TO RELATED APPLICATIONS

This application is a continuation of International Application No. PCT/JP2021/039888, filed on Oct. 28, 2021, which claims priority from Japanese Patent Application No. 2020-181879, filed on Oct. 29, 2020. The entire disclosure of each of the above applications is incorporated herein by reference.

### BACKGROUND

### Technical Field

The present disclosure relates to an information processing device, an information processing method, and an information processing program.

### Related Art

Hitherto, a technique for cleaning each region obtained by dividing the inside of a facility, such as a hospital, based on priority determined for each region is disclosed. For example, JP2009-532097A describes that a region where a possibility of infection is high is specified and a degree and a frequency of sterilization of the region are increased. Furthermore, for example, JP2014-529318A describes that surfaces of furniture and equipment in a room where there is a high probability that bacteria is present are disinfected with priority over a floor, a wall, and a ceiling in the room.

In recent years, there is a demand for a technique for performing cleaning depending on a state in a region. For example, it is known that there is an increasing possibility that an infectious disease spreads as people are densely populated, and to suppress the spread of the infectious disease, it is considered that a region where people are densely populated is preferably intensively cleaned. Note that, in the technique disclosed in JP2009-532097A and JP2014-529318A, it is not possible to perform cleaning depending on the state in the region.

### SUMMARY

The present disclosure provides an information processing device, an information processing method, and an information processing program capable of performing cleaning depending on a state of each region.

According to a first aspect of the present disclosure, there is provided an information processing device comprising at least one processor, in which the processor is configured to acquire presence information that is information indicating a state of presence of a person in each of a plurality of regions, for each region, and generate, for each of the plurality of regions, cleaning information for instructing cleaning of the region based on the presence information.

According to a second aspect of the present disclosure, in the first aspect, the presence information may include information indicating the number of people who are present in the region within a predetermined period.

According to a third aspect of the present disclosure, in the aspect, the presence information may include information indicating the total number of hours for which a person is present in the region within a predetermined period.

According to a fourth aspect of the present disclosure, in the second or third aspect, the presence information may be determined based on a protection state of a person who is present in the region.

According to a fifth aspect of the present disclosure, in the aspect, the cleaning information may include, as a content of an instruction pertaining to the cleaning of the region, information regarding at least one of a need for cleaning, a cleaning time, a cleaning measure, or an object to be cleaned.

According to a sixth aspect of the present disclosure, in the aspect, the plurality of regions may include regions that overlap other regions.

According to a seventh aspect of the present disclosure, in the aspect, division into the plurality of regions may be set in advance depending on a past record regarding the state of presence of a person.

According to an eighth aspect of the present disclosure, in the aspect, division into the plurality of regions may be set in advance depending on a degree of tolerance for contamination of the region.

According to a ninth aspect of the present disclosure, in the aspect, the processor may be configured to generate the cleaning information based on a degree of tolerance for contamination of the region, in addition to the presence information.

According to a tenth aspect of the present disclosure, in the aspect, the processor may be configured to generate the cleaning information based on a flow of air of the region, in addition to the presence information.

According to an eleventh aspect of the present disclosure, in the aspect, the processor may be configured to generate disinfection information for instructing disinfection based on the presence information, for each of a plurality of people who are respectively present in any of the plurality of regions.

According to a twelfth aspect of the present disclosure, in the aspect, the processor may be configured to perform control such that a display device displays the cleaning information to instruct the cleaning of the region based on the cleaning information.

According to a thirteenth aspect of the present disclosure, in the aspect, the processor may be configured to perform control such that a notification device notifies of the cleaning information to instruct the cleaning of the region based on the cleaning information.

According to a fourteenth aspect of the present disclosure, in the aspect, the processor may be configured to perform control such that a cleaning device executes the cleaning of the region based on the cleaning information.

According to a fifteenth aspect of the present disclosure, there is provided an information processing method by which a computer executes a process comprising acquiring presence information that is information indicating a state of presence of a person in each of a plurality of regions, for each region, and generating, for each of the plurality of regions, cleaning information for instructing cleaning of the region based on the presence information.

According to a sixteenth aspect of the present disclosure, there is provided an information processing program that causes a computer to execute a process comprising acquiring presence information that is information indicating a state of presence of a person in each of a plurality of regions, for each region, and generating, for each of the plurality of regions, cleaning information for instructing cleaning of the region based on the presence information.

According to the aspect, the information processing device, the information processing method, and the information processing program of the present disclosure of the present disclosure can perform cleaning depending on the state of each region.

### BRIEF DESCRIPTION OF THE DRAWINGS

FIG. 1 is a schematic configuration diagram of an information processing system.

FIG. 2 is a block diagram showing an example of a hardware configuration of an information processing device.

FIG. 3 is a block diagram showing an example of a functional configuration of the information processing device.

FIG. 4 is a diagram showing an example of a correspondence table for use in generating cleaning information.

FIG. 5 is a top view illustrating division of regions according to a specific example.

FIG. 6 is a diagram visualizing a log acquired by a log acquisition device.

FIG. 7 is a diagram showing an example of presence information and cleaning information according to the specific example.

FIG. 8 is a flowchart illustrating an example of information processing.

FIG. 9 is a top view illustrating division of regions according to a first modification example.

FIG. 10 is a diagram showing an example of presence information and cleaning information according to the first modification example.

FIG. 11 is a top view illustrating division of regions according to a second modification example.

FIG. 12 is a diagram showing an example of presence information and cleaning information according to the second modification example.

### DETAILED DESCRIPTION

Hereinafter, an embodiment for realizing the technique of the present disclosure will be described in detail referring to the drawings.

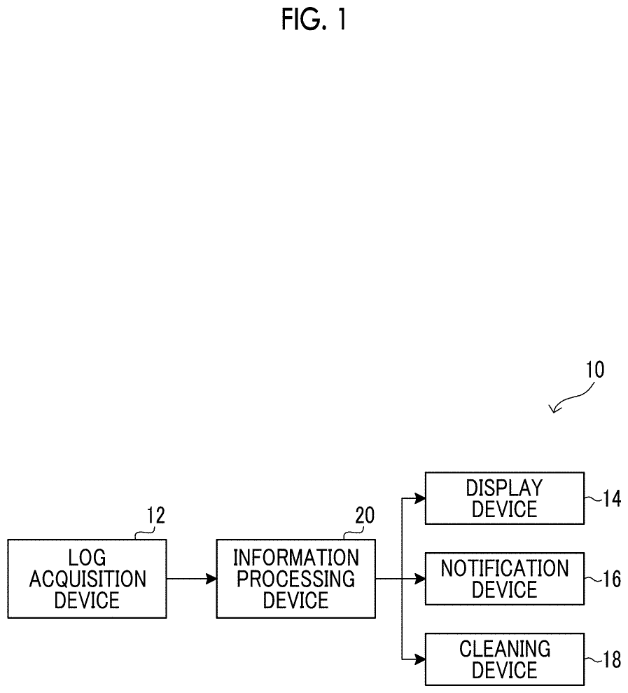

Referring to FIG. 1, an example of a configuration of an information processing system 10 according to the present exemplary embodiment will be described. As shown in FIG. 1, the information processing system 10 comprises a log acquisition device 12 and an information processing device 20. The information processing system 10 comprises at least one of a display device 14, a notification device 16, or a cleaning device 18. The information processing device 20 can perform communication with each of the log acquisition device 12, the display device 14, the notification device 16, and the cleaning device 18 through a network. In the present exemplary embodiment, an example where the log acquisition device 12, the information processing device 20, the display device 14, the notification device 16, and the cleaning device 18 are provided in a hospital will be described.

The log acquisition device 12 acquires a log that records entrance and exit of a person in each of a plurality of regions obtained by dividing the inside of the hospital in advance and transmits the acquired log to the information processing device 20. The log includes identification information indicating a person, identification information indicating a region where the person enters and exits, an entrance time, and an exit time. A person is, for example, a patient who visits the hospital, a companion of the patient, and a medical employee who works at the hospital.

As the log acquisition device 12, a known device and system that can acquire a log of entrance and exit of a person in each region can be applied. For example, a configuration in which an integrated circuit (IC) tag reader is provided for each region, and in a case where a person enters and leaves each region, the IC tag reader reads an IC tag carried with the person to acquire a log of entrance and exit of the person in each region along with identification information of the person may be used. The identification information of each person is given to the IC tag carried with the person.

Alternatively, for example, a configuration in which the inside of a region is imaged with a camera and image analysis processing is executed on a captured video to acquire a log of entrance and exit of a person in each region may be used. Furthermore, for example, a configuration in which a log of entrance and exit of a person in each region is acquired based on position information obtained using a portable terminal, a wearable terminal, a tag, or the like carried with a person may be used. As a measurement method of the position information, a known method using Bluetooth (Registered Trademark), a wireless local area network (LAN), a global positioning system (GPS), an ultrasonic wave, or the like can be applied.

The display device 14 is, for example, a device that can display information on a screen, such as a liquid crystal display and an organic electro luminescence (EL) device. The display device 14 may be provided, for example, at a place where a worker (hereinafter, referred to as a “cleaning worker”) who performs cleaning of each region can view the screen. The display device 14 may be a portable terminal carried with the cleaning worker. The notification device 16 is, for example, a device that can notify of information by lighting of a light emitting diode (LED) lamp, ringing of a buzzer, or the like. The notification device 16 may be provided for each object to be cleaned (for example, a ceiling, a wall, a floor, or a desktop) in each region. The cleaning device 18 is a device that can clean or sterilize a floor, a wall, a ceiling, a desktop, air, and the like in the hospital. As the cleaning device 18, for example, a moving device (for example, a traveling robot or a drone) having a cleaning measure, an ultraviolet irradiation device, a disinfectant solution spraying device, or an air purification device comprising a dust collection filter, such as a high efficiency particulate air (HEPA) filter, can be applied.

The information processing device 20 according to the present exemplary embodiment has a function of providing an instruction of cleaning to the cleaning worker and an instruction of execution of cleaning to the cleaning device 18 by the display device 14 and the notification device 16 depending on a state of presence of a person in each region. In general, it is known that, in a region where people are more densely populated and stay long, there is an increasing possibility that an infectious disease spreads. To suppress the spread of the infectious disease, it is considered that a region where people are densely populated and stay long are preferably intensively cleaned. Hereinafter, an example of the configuration of the information processing device 20 according to the present exemplary embodiment will be described.

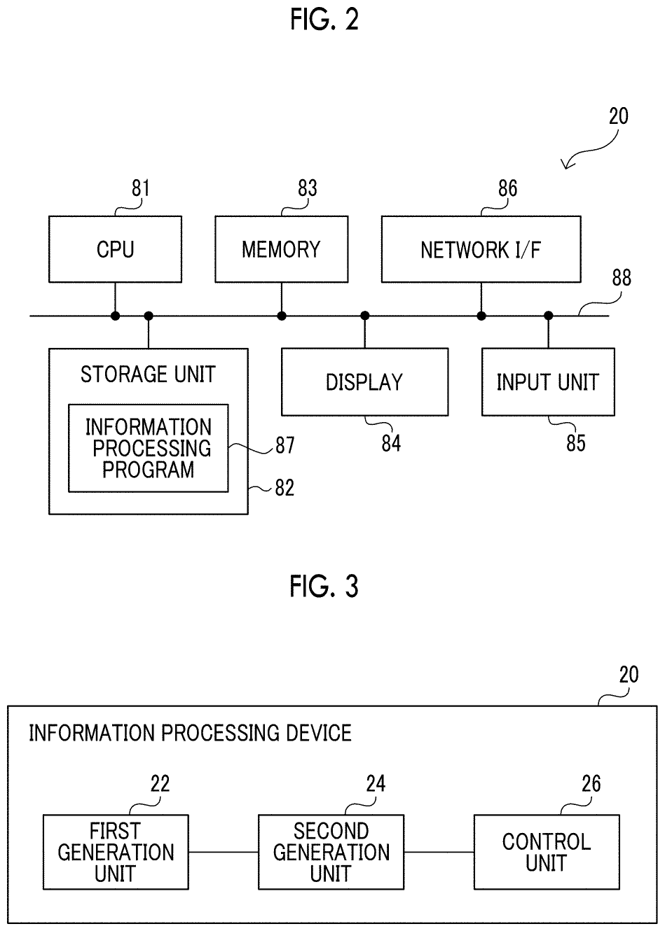

First, the hardware configuration of the information processing device 20 according to the present exemplary embodiment will be described referring to FIG. 2. As shown in FIG. 2, the information processing device 20 includes a central processing unit (CPU) 81, a non-volatile storage unit 82, and a memory 83 as a temporary storage area. The information processing device 20 includes a display 84, such as a liquid crystal display, an input unit 85, such as a keyboard and a mouse, and a network interface (I/F) 86 that is connected to a network. The CPU 81, the storage unit 82, the memory 83, the display 84, the input unit 85, and the network I/F 86 are connected to be able to transfer various kinds of information with each other through a bus 88, such as a system bus and a control bus.

The storage unit 82 is realized by a storage medium, such as a hard disk drive (HDD), a solid state drive (SSD), and a flash memory. An information processing program 87 is stored in the storage unit 82. The CPU 81 reads out the information processing program 87 from the storage unit 82, develops the information processing program 87 to the memory 83, and executes the developed information processing program 87. The CPU 81 is an example of a processor of the present disclosure.

Next, the functional configuration of the information processing device 20 according to the present exemplary embodiment will be described referring to FIG. 3. As shown in FIG. 3, the information processing device 20 includes a first generation unit 22, a second generation unit 24, and a control unit 26. The CPU 81 functions as the first generation unit 22, the second generation unit 24, and the control unit 26 by executing the information processing program 87.

The first generation unit 22 acquires the log transmitted from the log acquisition device 12, through the network I/F 86, and generates presence information that is information indicating a state of presence of a person in each of a plurality of regions, based on the log for each region.

The second generation unit 24 acquires the presence information of each region generated by the first generation unit 22, and generates, for each of a plurality of regions, cleaning information for instructing cleaning of the region based on the presence information. A generation method of the cleaning information is not particularly limited, and for example, a correspondence table in which the presence information and the cleaning information are associated in advance may be used or the cleaning information may be calculated by a mathematical expression for obtaining the cleaning information with the presence information as a variable.

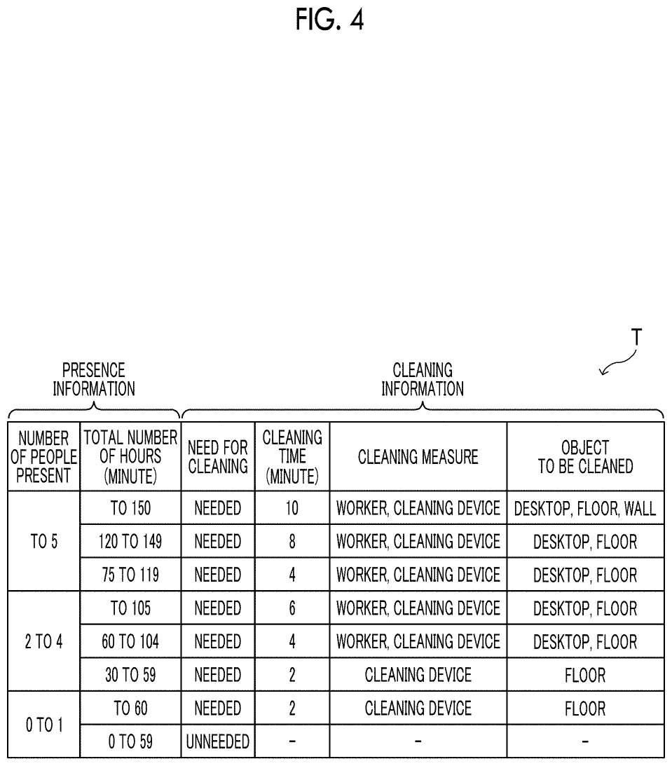

In the present exemplary embodiment, a configuration in which the second generation unit 24 generates the cleaning information by a method using a correspondence table T shown in FIG. 4 will be described. As shown in FIG. 4, in the correspondence table T, the cleaning information corresponding to the presence information is recorded in advance. The second generation unit 24 collates the presence information acquired from the first generation unit 22 with the correspondence table T to specify the cleaning information corresponding to the presence information for each region. The correspondence table T is stored in, for example, the storage unit 82.

As shown in FIG. 4, the presence information according to the present exemplary embodiment includes information indicating the number of people (hereinafter, referred to as “the number of people present”) who are present in a region within a predetermined period and the total number of hours for which a person is present in the region within the predetermined period. The predetermined period is, for example, a period from a point in time at which previous cleaning is performed to a start point in time of processing by the first generation unit 22. The total number of hours is a time obtained by accumulating a time (hereinafter, referred to as a “presence time”) from entrance to exit of one person in one region for all people who enter the region.

As shown in FIG. 4, the cleaning information according to the present exemplary embodiment includes information regarding at least one of a need for cleaning, a cleaning time, a cleaning measure, or an object to be cleaned, as contents of an instruction pertaining to cleaning of a region. The cleaning measure is, for example, a cleaning worker and the cleaning device 18. The object to be cleaned is, for example, a ceiling, a wall, a floor, a desktop, and air.

The control unit 26 controls at least one of the display device 14, the notification device 16, or the cleaning device 18 based on the cleaning information generated by the second generation unit 24. Specifically, the control unit 26 performs control such that the display device 14 displays the cleaning information to instruct cleaning of the region based on the cleaning information. The control unit 26 performs control such that the notification device 16 notifies of the cleaning information to instruct cleaning of the region based on the cleaning information. The control unit 26 performs control such that the cleaning device 18 executes cleaning of the region based on the cleaning information.

The control unit 26 may end the control of the display device 14, the notification device 16, and the cleaning device 18 in a case where cleaning of the region to which cleaning is instructed is completed. The completion of cleaning by the cleaning worker can be detected, for example, through image processing on a video obtained by imaging with a camera. The completion of cleaning by the cleaning device 18 can be detected with reception of a signal indicating the completion of cleaning from the cleaning device 18. The control unit 26 may perform control such that, in a case where cleaning is not completed within a predetermined period after cleaning is instructed, the display device 14 and the notification device 16 notify that cleaning is not completed within the predetermined period after cleaning is instructed.

[Specific Example]

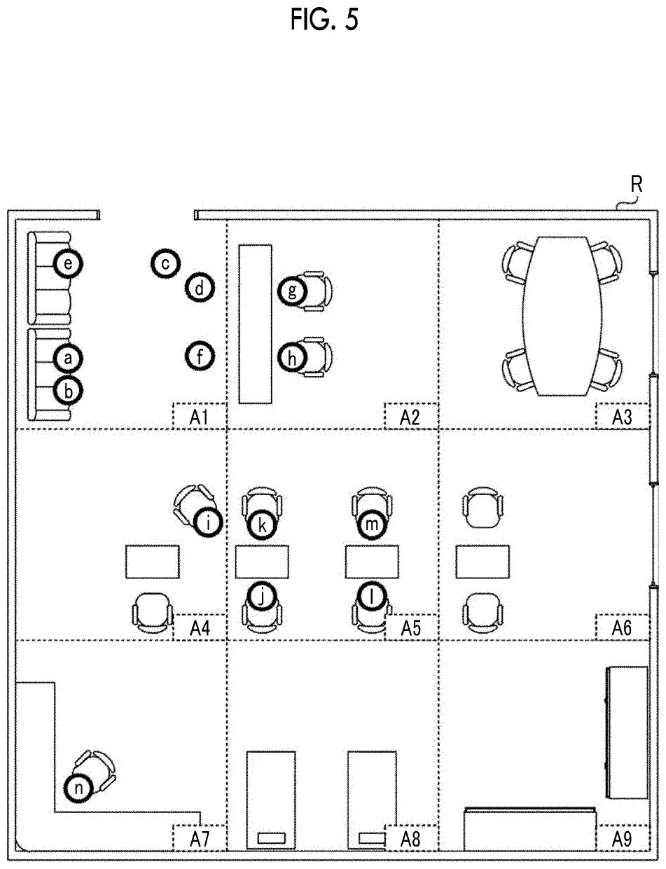

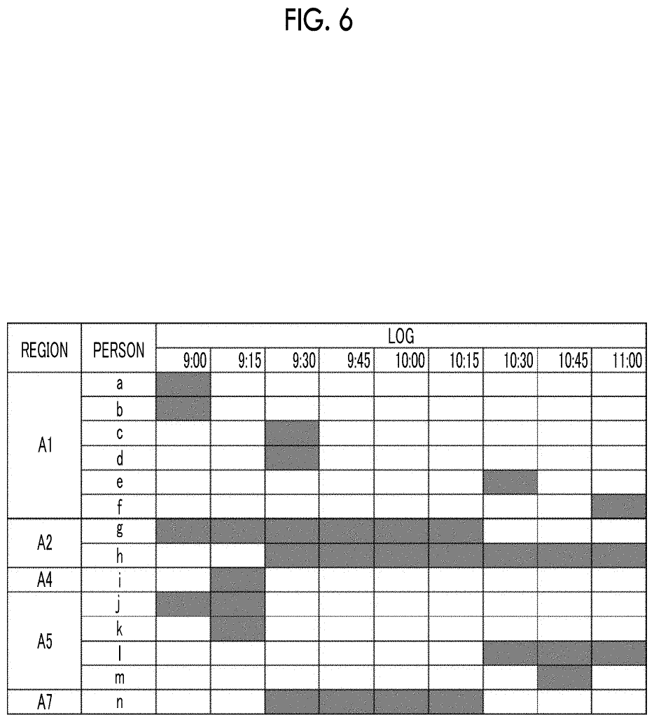

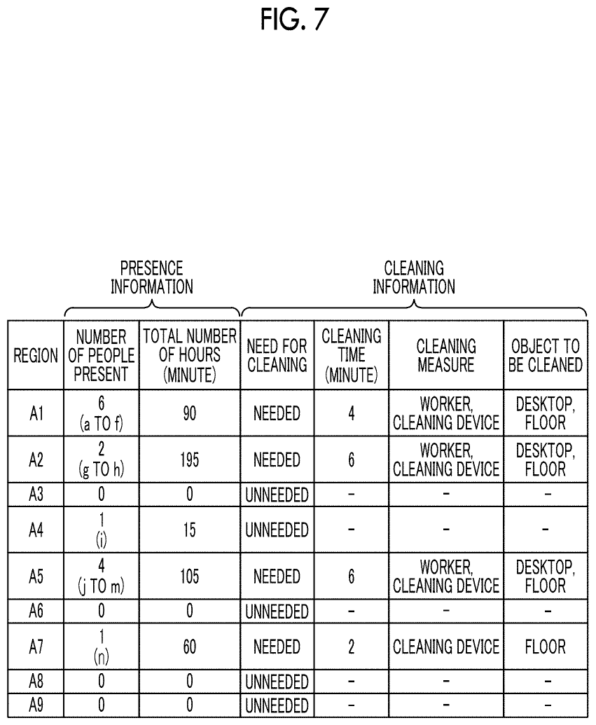

A specific example of processing by the first generation unit 22, the second generation unit 24, and the control unit 26 described above will be described referring to FIGS. 5 to 7. FIG. 5 is a top view showing a specific example of one room R in a hospital. As shown in FIG. 5, in regard to a plurality of regions in the present specific example, the room R is divided into nine regions A1 to A9. In FIG. 5, persons a to n are shown by circles. FIG. 6 is a diagram visualizing a log of entrance and exit of the persons a to n in the respective regions A1 to A9. In FIG. 6, presence times from entrance to exit of the persons a to n in the regions A1 to A9 are shown by black portions. FIG. 7 is a diagram showing presence information and cleaning information for each of the regions A1 to A9, generated by the first generation unit 22 and the second generation unit 24.

As an example, processing of generating the presence information and the cleaning information for the region A1 will be described. The first generation unit 22 calculates the number of people present of the region A1 within a predetermined period (9:00 to 11:15) to be six (persons a to f), based on the log (see FIG. 6). The first generation unit 22 calculates the total number of hours of the region A1 within the predetermined period (9:00 to 11:15) to be 90 minutes, based on the log (see FIG. 6). As shown in FIG. 7, the first generation unit 22 generates information including the values as the presence information of the region A1.

The second generation unit 24 acquires the presence information generated by the first generation unit 22 and collates the presence information with the correspondence table T (see FIG. 4). Since the number of people present of the region A1 is six, and the total number of hours is 90 minutes, the second generation unit 24 generates the cleaning information (the need for cleaning is “needed”, the cleaning time is “4 minutes”, the cleaning measure is “worker, cleaning device”, and the object to be cleaned is “desktop, floor”) in the third line from the top of the correspondence table T (see FIG. 4), as the cleaning information of the region A1.

The control unit 26 performs control such that the display device 14 displays a message “Please clean the desktop in the region A1 for 4 minutes.” based on the cleaning information, thereby instructing cleaning of the desktop to the cleaning worker. The control unit 26 causes the notification device 16 to give notification meaning cleaning of the region A1, thereby instructing cleaning to the cleaning worker. The control unit 26 performs control such that the cleaning device 18 cleans the floor of the region A1 for 4 minutes.

Processing by the first generation unit 22, the second generation unit 24, and the control unit 26 is executed in the same procedure for other regions A2 to A9.

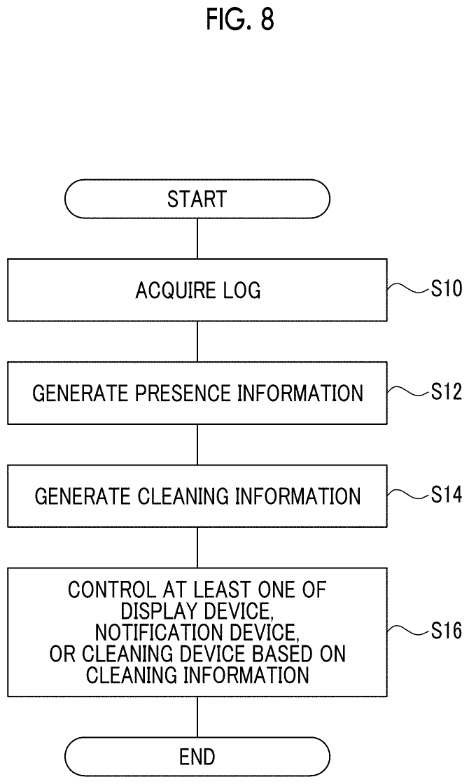

Next, the operations of the information processing device 20 according to the present exemplary embodiment will be described referring to FIG. 8. The CPU 81 executes the information processing program 87, whereby information processing shown in FIG. 8 is executed. The information processing shown in FIG. 8 is executed, for example, at a predetermined timing as a timing at which cleaning can be performed, such as a break time.

In Step S10 of FIG. 8, the first generation unit 22 acquires the log transmitted from the log acquisition device 12 through the network I/F 86. In Step S12, the first generation unit 22 generates presence information regarding each of a plurality of regions based on the log acquired in Step S10.

In Step S14, the second generation unit 24 acquires the presence information generated in Step S12, and generates cleaning information regarding each of a plurality of regions based on the presence information. In Step S16, the control unit 26 controls at least one of the display device 14, the notification device 16, or the cleaning device 18 based on the cleaning information generated in Step S14.

As described above, the information processing device according to the present exemplary embodiment comprises at least one processor, and the processor is configured to acquire presence information that is information indicating a state of presence of a person in each of a plurality of regions, for each region, and generate, for each of a plurality of regions, cleaning information for instructing cleaning of the region based on the presence information. Accordingly, since cleaning depending on the state of each region can be performed, it is possible to intensively clean a region where there is a high possibility that an infectious disease spreads, and it is advantageous to suppress the spread of the infectious disease. It is possible to omit cleaning for a region where there is less possibility that the infectious disease spreads, and it is advantageous to improve efficiency of cleaning work.

In the above-described exemplary embodiment, a method of division into a plurality of regions is not particularly limited. For example, one room in a hospital may be divided into a plurality of regions (see FIG. 5), one room may be set as one region, or a plurality of room may be collectively set as one region. For example, an approach of zoning, cohorting, or the like in a hospital may be applied, and the division into a plurality of regions may be set in advance depending on the degree of tolerance for region contamination.

Another example of the method of division into a plurality of regions will be described with reference to modification examples of the above-described specific example.

### First Modification Example

In the above-described specific example, as shown in FIG. 7, cleaning information that cleaning of the region A4 is unneeded is generated. In contrast, in the region A4, since the person i is present at a position close to the person k and the person j who are present in the region A5 where cleaning is needed, it is considered that cleaning is preferably performed for a region neighboring the region A5 where the person i is present, in the region A4.

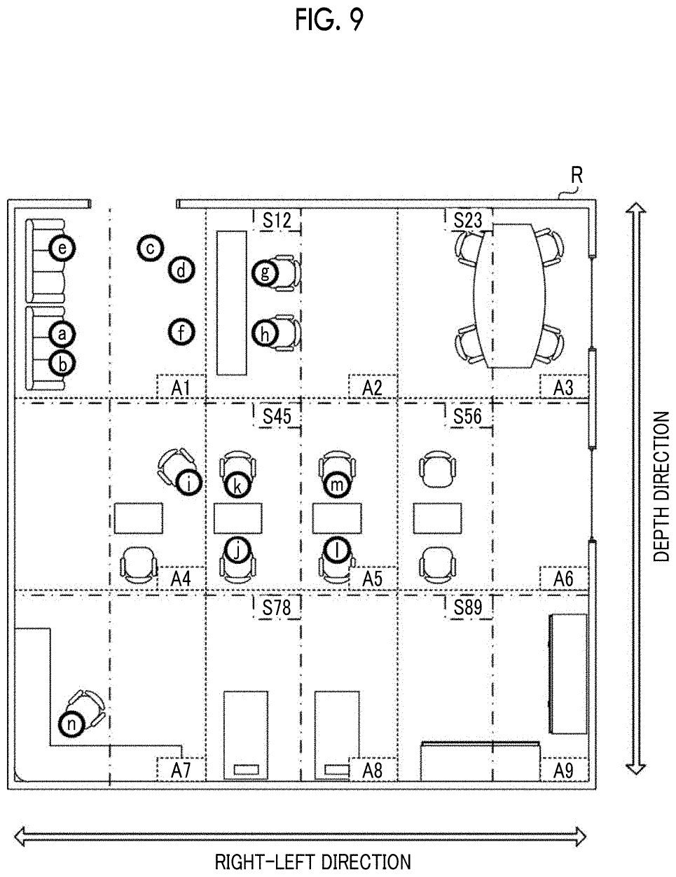

To perform cleaning including such a region, a plurality of regions in the present exemplary embodiment may include a region (hereinafter, referred to an overlapping regions) that overlaps another region. In FIG. 9, in addition to regions A1 to A9 in the same room R as in FIG. 5, overlapping regions S12, S23, S45, S56, S78, and S89 that stride over two adjacent regions in a right-left direction among the regions A1 to A9 are shown by one-dot chain lines. Hereinafter, the regions A1 to A9 are referred to as “regions A” in a case where there is no distinction therebetween, and the overlapping regions S12, S23, S45, S56, S78, and S89 are referred to as “overlapping regions S” in a case where there is no distinction therebetween.

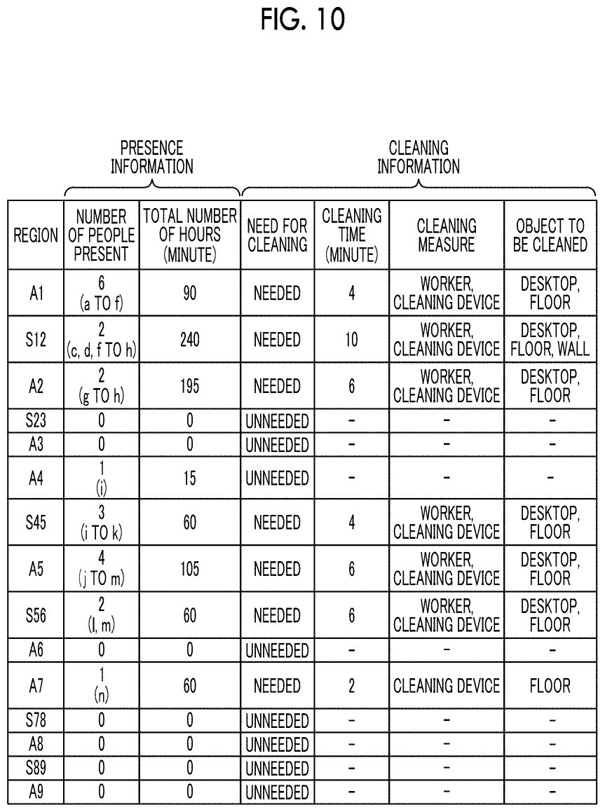

In this case, the first generation unit 22 and the second generation unit 24 generate presence information and cleaning information for the overlapping region S, in addition to the regions A. FIG. 10 shows presence information and cleaning information that are generated based on the correspondence table T of FIG. 4 and the log of FIG. 6, for each of the regions A and the overlapping regions S shown in FIG. 9. The control unit 26 performs various kinds of control while employing the cleaning information in which the cleaning time is longer and there are a lot of cleaning measures and cleaning locations, among the cleaning information generated for each of the regions A and the overlapping regions S, for the regions where the regions A and the overlapping regions S overlap.

For example, the control unit 26 performs various kinds of control based on the cleaning information of the overlapping region S45 in which the cleaning time is longer and there are a lot of cleaning measures and cleaning locations, for a region where the region A4 and the overlapping region S45 overlap. According to such a configuration, it is possible to generate appropriate cleaning information for a neighboring region, in addition to a region where there is a high possibility that an infectious disease spreads. Therefore, it is possible to further suppress the spread of the infectious disease.

In the present modification example, for simplification of description, although a configuration in which the overlapping regions that stride over two adjacent regions in the right-left direction among the region regions A1 to A9 are included has been described, overlapping regions that stride over two adjacent regions in a depth direction among the regions A1 to A9 are more preferably included.

### Second Modification Example

In the above-described specific example, as shown in FIG. 5, an example where the room R is equally divided into nine parts has been described. Note that, for example, in performing cleaning in the intervals of regular business of a hospital, in a case where a region to be cleaned is wide, it may be difficult to secure a cleaning time. Therefore, for example, for a region where it is known that cleaning is to be intensively performed, from a past record, at a location where a lot of people enter and leave, a location where a person stays long, or the like, it is considered that the region is preferably further divided.

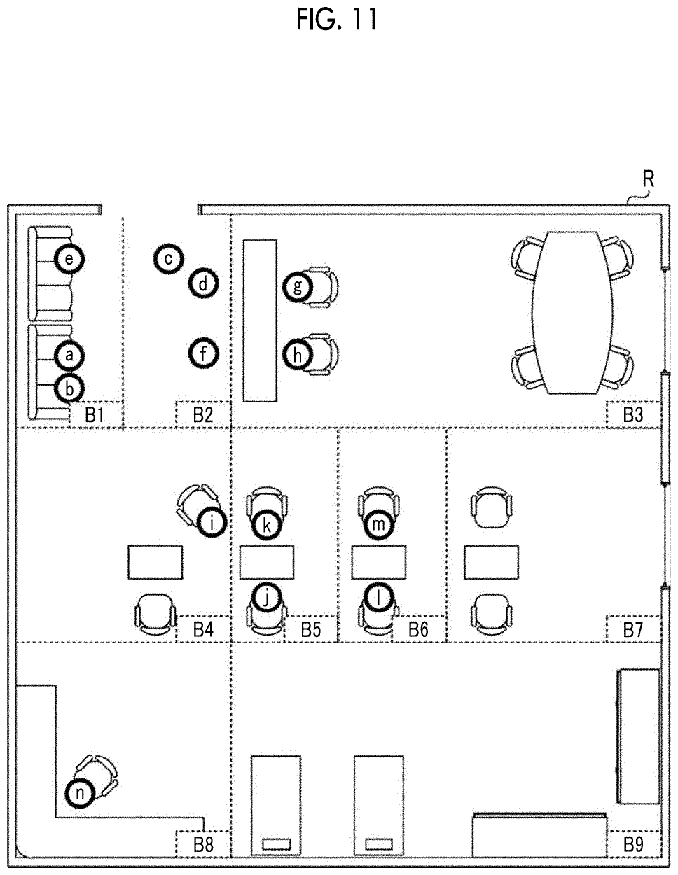

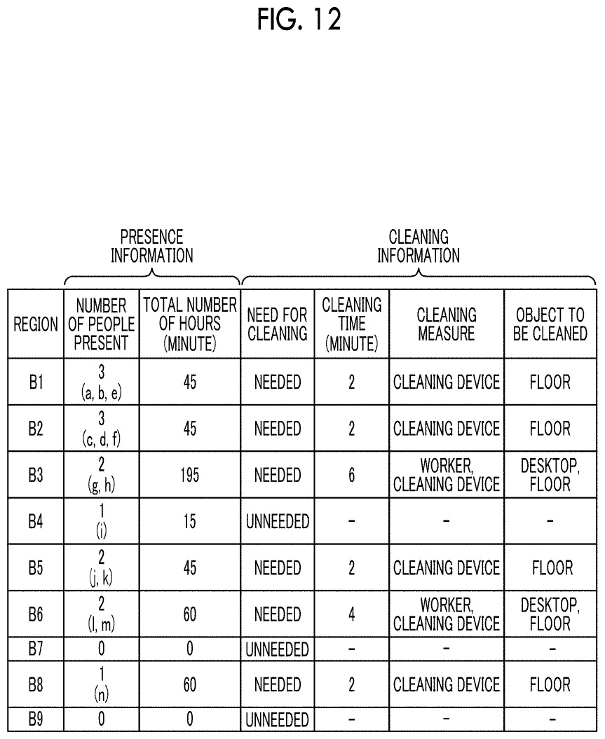

Accordingly, the division into a plurality of regions in the present exemplary embodiment may be set in advance depending on past records regarding a state of presence of a person. FIG. 11 shows an example where the same room R as in FIG. 5 is divided into regions B1 to B9 depending on past records regarding the state of presence of a person. FIG. 12 shows presence information and cleaning information that are generated based on the correspondence table T of FIG. 4 and the log of FIG. 6, for the example of FIG. 11.

for example, while the regions B5 and B6 correspond to regions obtained by further dividing the region A5 of FIG. 5, as shown in FIG. 12, different cleaning information are generated depending on presence information pertaining to the respective regions. Accordingly, in executing cleaning of the region B5, since it is possible to perform cleaning with contents appropriate for the region B5 without interfering with another work performed in the region B6, it is advantageous to improve efficiency of cleaning work.

In the above-described exemplary embodiment, the presence information may be determined based on a protection state of a person who is present in a region. This is because it is considered that a protected person is less likely to spread an infectious disease than an unprotected person. The protection state is determined, for example, depending on a wearing state of a mask and protective clothing, and an execution state of hand washing, gargling, and disinfection with alcohol. The presence information is determined based on the protection state of the person who is present in the region, whereby it is advantageous to improve efficiency of cleaning work while suppressing the spread of the infectious disease as appropriate.

In this case, for example, the first generation unit 22 may make weights of the number of people present and the presence time different depending on the presence or absence of wearing of the mask, such as counting the number of people present while counting a person wearing the mask as 0.5 persons. Alternatively, the first generation unit 22 may make the weights of the number of people present and the presence time different depending on the type of the mask, such as a non-woven fabric mask, a gauze mask, a surgical mask, and a particulate mask. The presence or absence of wearing of the mask of the person and the type of the mask can be determined based on, for example, an image obtained by imaging the person with a camera.

In this case, for example, the first generation unit 22 may make the weights of the number of people present and the presence time different depending on the execution state of hand washing, such as counting the number of people present while counting a person who insufficiently wash the hands, as 1.5 persons. Determination about whether or not hand washing is sufficient can be performed based on whether or not a time determined as a time during which the person is washing the hands is equal to or greater than a predetermined threshold value, by a function of a wearable terminal, such as a smart watch.

In the above-described exemplary embodiment, although a configuration in which the presence information includes information indicating the number of people present and the total number of hours has been described, the technique of the present disclosure is not limited thereto. For example, a configuration in which the presence information includes only one of the number of people present and the total number of hours may be used.

In the above-described exemplary embodiment, the first generation unit 22 may track movement of a person between regions, and in a case where a person who exits a certain region returns to the same region again, may generate presence information in such a manner that the number of people present of the region is not counted twice. This is because it is considered that the same person returns to the same region and an infectious disease is less likely to further spread. The presence information not including the same person in duplicate is generated, whereby it is possible to perform cleaning more conforming to the state of each region. Tracking movement of a person between regions is also advantageous to specify an infection route. As a method for tracking movement of a person between regions, for example, a known method, such as a method using identification information of an IC tag and a method using face recognition by a camera, can be applied.

In the above-described exemplary embodiment, the second generation unit 24 may generate the cleaning information in consideration of various kinds of information, in addition to the presence information. For example, the second generation unit 24 may generate the cleaning information based on the degree of tolerance for region contamination, in addition to the presence information. This is because cleaning is preferably performed with a smaller number of people present and a smaller total number of hours in a region where advanced cleanliness is required, such as an operation room, compared to a region where cleanliness is not much required, such as a waste treatment chamber. The second generation unit 24 may generate the cleaning information using a plurality of correspondence tables T that are different for each degree of tolerance for region contamination. A plurality of correspondence tables T that are different for each degree of tolerance for region contamination are stored in the storage unit 82 in advance. The degree of tolerance for region contamination is determined by, for example, an approach of zoning, cohorting, or the like in a hospital. According to such a configuration, it is possible to perform cleaning depending on the degree of tolerance for region contamination, in addition to the state of each region.

For example, the second generation unit 24 may generate the cleaning information based on a flow of air in a region, in addition to the presence information. This is because it is considered that a virus causing an infectious disease is highly likely to remain on a downstream side rather than an upstream side in a flow of air formed by air conditioning, a window, and a ventilating opening, and the like. That is, it is considered that the infectious disease is more highly likely to spread in a region on the downstream side than in a region on the upstream side, and cleaning is preferably performed with a smaller number of people present and a smaller total number of hours. Accordingly, in a case where a flow of air is formed over a plurality of regions, the second generation unit 24 may generate the cleaning information using a plurality of correspondence tables T that are different for each flow of air, such as an upstream side and a downstream side. A plurality of correspondence tables T that are different for each flow of air are stored in the storage unit 82 in advance. As a method for detecting a flow of air, for example, a known method, such as a method using an infrared camera and a particle image velocimeter, can be applied. According to such a configuration, it is possible to perform cleaning depending on a flow of air of a region, in addition to the state of each region.

For example, the second generation unit 24 may generate the cleaning information based on an area of a region, in addition to the presence information. This is because cleaning is preferably performed with a smaller number of people present and a smaller total number of hours as the area is smaller. Specifically, in a case where the area of each region is different as described in the above-described second modification example, the second generation unit 24 may generate the cleaning information using a plurality of correspondence tables T that are different for each area. A plurality of correspondence tables T that are different for each area are stored in the storage unit 82 in advance. According to such a configuration, it is possible to perform cleaning more conforming to the state of each region.

In the above-described exemplary embodiment, the second generation unit 24 may generate disinfection information for instructing disinfection to each of a plurality of people who are present in each of a plurality of regions, based on the presence information. “Disinfection” is, for example, hand washing, gargling, disinfection with alcohol, and irradiation of ultraviolet light having a wavelength of 222 nm that has high safety to a human body and is capable of inactivating a virus. Specifically, the second generation unit 24 generates disinfection information of contents for instructing execution of disinfection for a person who is present in a region (hereinafter, referred to as a “region to be disinfected”) where each of the number of people present and the total number of hours is equal to or greater than a predetermined threshold value. In this case, the control unit 26 performs control such that the display device 14 and the notification device 16 instruct disinfection of a person based on the disinfection information generated by the second generation unit 24. According to such a configuration, it is possible to further suppress the spread of an infectious disease.

In this case, in a case where a person who is present in the region to be disinfected moves to another region that is not a region to be disinfected, the second generation unit 24 may generate disinfection information of contents for instructing execution of disinfection for another person who is present in the region after the movement. In a case where a person who is present in the region to be disinfected moves to another region that is not a region to be disinfected, the second generation unit 24 may generate cleaning information of contents for instructing execution of cleaning for the region after the movement. According to such configurations, even in a region that is not to be a target of disinfection alone, in a case where a virus is likely to be introduced from the region to be disinfected, since it is possible to perform disinfection of a person who is present in the region and cleaning of the region, it is possible to further suppress the spread of an infectious disease.

In this case, the second generation unit 24 may generate disinfection information in consideration of a speaking time and a speaking amount of a person in a region, an orientation of a face with respect to a flow of air, a degree of approach to another person, and the like. Such information can be acquired based on, for example, an image obtained by imaging a person with a camera. According to such a configuration, it is possible to further suppress the spread of an infectious disease.

In the above-described exemplary embodiment, the control unit 26 may perform control for instructing cleaning in consideration of a margin, for a region where cleaning is designated by the cleaning information. For example, in addition to the region where cleaning is designated, cleaning may be instructed for another region adjacent to the region. For example, cleaning may be instructed for a region obtained by enlarging a region where cleaning is designated, by a predetermined area. According to such a configuration, it is possible to further suppress the spread of an infectious disease.

In the above-described exemplary embodiment, the control unit 26 may perform control such that, in a case where it is detected that cleaning is likely to be needed, based on the cleaning information, the display device 14 and the notification device 16 notify that cleaning is likely to be needed. For example, the control unit 26 may notify of a message “This region is crowded, and then, is to be a target of cleaning before long”. Detection about whether or not cleaning is likely to be needed can be performed depending on whether or not the total number of hours is within a predetermined range from a lower limit of the total number of hours at which the need for cleaning is determined to be “needed” in the correspondence table T shown in FIG. 4. According to such a configuration, it is possible to serve a timing at which a person executes a method for suppressing the spread of the infectious disease, such as restriction on exit from a region and entrance to a region, isolation of a person, and execution of ventilation and cleaning, and it is advantageous to suppress the spread of an infectious disease.

In the above-described exemplary embodiment, although a configuration in which the information processing device 20 is applied to the hospital has been described, the technique of the present disclosure is not limited thereto. The information processing device 20 according to the present exemplary embodiment can be applied to various facilities, such as an office, a school, an accommodation facility, an eating facility, a nursing facility, and a refugee facility.

In the above-described exemplary embodiment, although a configuration in which the first generation unit 22 of the information processing device 20 generates the presence information, the technique of the present disclosure is not limited thereto. For example, a configuration in which the log acquisition device 12 or any external device generates the presence information and transmits the presence information to the information processing device 20 through a network may be used.

In the above-described exemplary embodiment, as the hardware structure of processing units that execute various kinds of processing, such as the first generation unit 22, the second generation unit 24, and the control unit 26, various processors described below can be used. Various processors include a programmable logic device (PLD) that is a processor capable of changing a circuit configuration after manufacturing, such as a field programmable gate array (FPGA), a dedicated electric circuit that is a processor having a circuit configuration dedicatedly designed for executing specific processing, such as an application specific integrated circuit (ASIC), and the like, in addition to a CPU that is a general-purpose processor that executes software (program) to function as various processing units, as described above.

One processing unit may be configured with one of various processors described above or may be configured with a combination of two or more processors (for example, a combination of a plurality of FPGAs or a combination of a CPU and an FPGA) of the same type or different types. A plurality of processing units may be configured with one processor.

As an example where a plurality of processing units are configured with one processor, first, as represented by a computer, such as a client or a server, a configuration in which one processor is configured with a combination of one or more CPUs and software, and the processor functions as a plurality of processing units is known. Second, as represented by System on Chip (SoC) or the like, a configuration in which a processor that realizes the functions of the entire system including a plurality of processing units by one integrated circuit (IC) chip is used is known. In this way, various processing units are configured using one or more processors among various processors described above as the hardware structure.

In addition, as the hardware structure of various processors is, more specifically, an electric circuit (circuitry), in which circuit elements, such as semiconductor elements, are combined can be used.

In the above-described exemplary embodiment, although an aspect where the information processing program 87 is stored (installed) in the storage unit 82 has been described, the technique of the present disclosure is not limited thereto. The information processing program 87 may be provided in a form of being recorded on a recording medium, such as a compact disc read only memory (CD-ROM), a digital versatile disc read only memory (DVD-ROM), and a universal serial bus (USB) memory. Alternatively, a configuration in which the information processing program 87 is downloaded from an external device through a network may be used. The technique of the present disclosure also extends to a storage medium that non-transitorily stores the information processing program, in addition to the information processing program.

The technique of the present disclosure can also combine the above-described embodiment as appropriate. The content of the above description and the content of the drawings are detailed description of portions according to the technique of the present disclosure, and are merely examples of the technique of the present disclosure. For example, description regarding the above configurations, functions, operations, and advantages are description regarding an example of the configurations, functions, operations, and advantages of the portions according to the technique of the present disclosure. Thus, it is needless to say that unnecessary portions may be deleted, new elements may be added, or replacements may be made in the above-described contents and the illustrated contents without departing from the gist of the technique of the present disclosure.

The entire disclosure of JP2020-181879, filed Oct. 29, 2020, is incorporated into the present specification by reference. All documents, patent applications, and technical standards described in the present specification are incorporated in the specification by reference to the same extent as in a case where the individual document, patent application, or technical standard is specifically and individually indicated to be incorporated by reference.

What is claimed is:

1. An information processing device comprising at least one processor, wherein the at least one processor is configured to:
acquire presence information that is information indicating a state of presence of a person in each of a plurality of regions, for each region; and
generate, for each of the plurality of regions, cleaning information for instructing cleaning of the region based on the presence information.

2. The information processing device according to claim 1, wherein the presence information includes information indicating the number of people who are present in the region within a predetermined period.

3. The information processing device according to claim 1, wherein the presence information includes information indicating the total number of hours for which a person is present in the region within a predetermined period.

4. The information processing device according to claim 2, wherein the presence information is determined based on a protection state of a person who is present in the region.

5. The information processing device according to claim 1, wherein the cleaning information includes, as a content of an instruction pertaining to the cleaning of the region, information regarding at least one of a need for cleaning, a cleaning time, a cleaning measure, or an object to be cleaned.

6. The information processing device according to claim 1, wherein the plurality of regions include regions that overlap other regions.

7. The information processing device according to claim 1, wherein division into the plurality of regions is set in advance depending on a past record regarding the state of presence of a person.

8. The information processing device according to claim 1, wherein division into the plurality of regions is set in advance depending on a degree of tolerance for contamination of the region.

9. The information processing device according to claim 1, wherein the at least one processor is configured to generate the cleaning information based on a degree of tolerance for contamination of the region, in addition to the presence information.

10. The information processing device according to claim 1, wherein the at least one processor is configured to generate the cleaning information based on a flow of air of the region, in addition to the presence information.

11. The information processing device according to claim 1, wherein the at least one processor is configured to generate disinfection information for instructing disinfection based on the presence information, for each of a plurality of people who are respectively present in any of the plurality of regions.

12. The information processing device according to claim 1, wherein the at least one processor is configured to perform control such that a display device displays the cleaning information to instruct the cleaning of the region based on the cleaning information.

13. The information processing device according to claim 1, wherein the at least one processor is configured to perform control such that a notification device notifies of the cleaning information to instruct the cleaning of the region based on the cleaning information.

14. The information processing device according to claim 1, wherein the at least one processor is configured to perform control such that a cleaning device execute the cleaning of the region based on the cleaning information.

15. An information processing method by which a computer executes a process comprising:
acquiring presence information that is information indicating a state of presence of a person in each of a plurality of regions, for each region; and
generating, for each of the plurality of regions, cleaning information for instructing cleaning of the region based on the presence information.

16. A non-transitory computer-readable storage medium storing an information processing program that causes a computer to execute a process comprising:
acquiring presence information that is information indicating a state of presence of a person in each of a plurality of regions, for each region; and
generating, for each of the plurality of regions, cleaning information for instructing cleaning of the region based on the presence information.
